(19) United States
(12) Patent Application Publication
(10) Pub. No.: US 2002/0164381 A1
(43) Pub. Date: Nov. 7, 2002

(54) Mitocidal compositions and methods

(75) Inventors: Jonah Shacknai, Scottsdale, AZ (US); Eugene H. Gans, Westport, CT (US)

Correspondence Address: William J. McNichol, Jr., Reed Smith LLP, 2500 One Liberty Place, 1650 Market Street, Philadelphia, PA, 19103-7301, US

(73) Assignee: Medicis Pharmaceutical Corporation

(21) Appl. No.: 10/022,476

(22) Filed: Dec. 18, 2001

Related U.S. Application Data
(63) Continuation-in-part of application No. 09/607,881, filed on Jun. 30, 2000.

(51) Int. Cl. A61K 7/075; A61K 33/04

(52) U.S. Cl. 424/703; 424/70.27

(57) ABSTRACT

Methods for the treatment and prevention of cutaneous mites and for the treatment and prevention of cutaneous inflammation of mammalian skin associated with cutaneous mite infestations utilizing compositions comprising sulfur, one or more sulfur derivative and a dermatologically acceptable carrier. The composition is preferably a cleanser.

### RELATED APPLICATIONS

This application is a continuation-in-part of Ser. No. 09/607,881 filed Jun. 30, 2000.

### FIELD OF THE INVENTION

The present invention relates to compositions and methods for the treatment (including, but not limited to the partial reduction) and prevention of infestations of the skin of mammals, particularly humans. These mites are usually in the order Acarina, and include all cutaneous mites, including but not limited to follicle mites, food mites, fowl mites, grain mites, harvest mites, murine mites, and scabies. The invention especially relates to mites of the genus Demodex.

### BACKGROUND OF THE INVENTION

Cutaneous mites in the order Acarina are often present in the epidermis, including the pilosebaceous infundibulum, and sometimes in the stratum corneum and the dermis. Demodex mites, particularly Demodex folliculorum and Demodex brevis, can be detected in sebaceous glands and hair follicles by skin biopsy. Small populations of mites normally reside in human skin, but an excess of mites or particular sensitivity to mites causes irritation to the skin. The irritation can exacerbate other skin disorders, including but not limited to acne rosacea and acne vulgaris. It has been thought that mites may contribute significantly to the cutaneous inflammation in skin disorders. Reduction of mites has been associated with a reduction in skin inflammation, and mite reduction is considered to be of therapeutic advantage.

Cutaneous inflammation from mites tends to extend into the deep dermis and surrounds vessels with some extension into dermal collagen. With the exception of scabies (Sarcoptes scabiei var. humanus), insect parts are rarely seen within the stratum corneum.

While Demodex mites burrow into the skin, other nonscabetic mites generally do not. The treatment of non-Demodex, nonscabetic infestations can conventionally consist of a warm soapy bath. Other mite treatments, including scabies treatments in the prior art, have included permethrin cream followed by lindane and sometimes sulfur. Additionally, pretreatment with keratolytic agents has also been used. Glucocorticoid administration has been used to mask symptoms and signs of scabies, but the infestation persists and the mites are still transmittable.

The art tends to avoid sulfur treatments, and currently favored therapies are pesticide treatments, such as permethrin and lindane. However, pesticide treatment has disadvantages. Many of these treatments are primarily useful for head lice treatment, which continue to develop resistance to pesticides. Second, concerns over the long-term effects of exposure to pesticides on human health have been raised.

Sulfur treatments for mites have been thought to be undesirable because they are odorous and therefore patient compliance is not optimal. An additional challenge for sulfur-based mitocides is depositing sufficient sulfur and sulfur derivatives to the skin. Cleansers are particularly challenging because depositing those active substances is difficult, given a cleanser's short time of contact with the skin and the inherent tendency of cleaning and rinsing actions to discourage depositions of a active substance.

There has been a long-felt need for an alternative effective mitocide composition.

### SUMMARY OF THE INVENTION

The present invention is directed to methods for the treatment and prevention of cutaneous mite infestation, and for the treatment and prevention of cutaneous inflammation of mammalian skin associated with cutaneous mite infestation, as well as for enhancing absorption of sulfur and sulfur derivatives into mammalian skin by the topical application of a composition comprising sulfur, one or more sulfur derivatives and a dermatologically acceptable carrier, preferably with a pH of from about 6.5 to about 8.1.

An embodiment of the invention provides an effective mitocide composition for the treatment (treatment is herein defined to include but is not limited to partial reduction) and prevention of cutaneous mites and for the treatment and prevention of cutaneous inflammation of mammalian skin associated with cutaneous mite infestations.

One embodiment is a topical solution in a cream form composition which comprises sulfur, one or more sulfur derivatives, and one or more dermatologically acceptable carriers, preferably having a pH of from about 6.5 to about 8.1. A preferred embodiment comprises water, sulfacetamide sodium, propylene glycol, isopropyl myristate, sulfur, light mineral oil, polysorbate 60, sorbitan monostearate, cetyl alcohol, hydrogenated cocoglycerides, stearyl alcohol, ordenone, benzyl alcohol, glyceryl stearate & PEG 100 stearate, zinc ricinoleate, dimethicone, xanthan gum, disodium EDTA, and sodium thiosulfate.

Another embodiment comprises sodium methyl oleyltaurate, disodium oleamido MEA sulfosuccinate, PEG-55 propylene glycol oleate, water, sodium cocoyl isethionate, methyl paraben, propyl paraben, disodium EDTA, cetyl alcohol, stearyl alcohol, glyceryl stearate & PEG-100 stearate, BHT, sodium thiosulfate, sulfacetamide sodium, magnesium aluminum silicate, xantham gum and sulfur.

Another embodiment is a cleanser composition which deposits a sufficient amount of sulfur and/or sulfur derivatives, which may have been converted into sulfur derivatives on the skin surface, at and below the stratum corneum. A sufficient amount of sulfur and/or sulfur derivatives is preferably delivered to one or more layers below the stratum corneum, including but not limited to epidermis, and dermis, in an especially preferred embodiment more than about 25% (all precentages given are weight percentages) of the retained dosage on the skin after application and any rinsing is absorbed.

Another embodiment is a high sorption composition which comprises sulfur, one or more sulfur derivatives, and one or more dermatologically acceptable carriers. A preferred embodiment includes a composition comprising water, xanthan gum, magnesium aluminum silicate, kaolin, silicone dioxide, sodium sulfacetamide, sodium thiosulfate, glyceryl stearate, PEG-100 stearate, quillaia saponaria extract, benzyl alcohol, and sulfur.

### DETAILED DESCRIPTION OF THE PREFERRED EMBODIMENTS

The present invention is a method of treatment and prevention of cutaneous mite infestations, especially mites of the order Acarina on mammals, preferably selected from the group consisting of dogs, cats and humans, and most preferably humans. Further the present invention provides for the treatment and prevention of cutaneous inflammation of mammalian skin, preferably human skin associated with cutaneous mite infestations. This includes treatment and prevention of cutaneous inflammations associated with skin disorders, including but not limited to acne rosacea. Surprisingly, the present invention also provides a method for treating and preventing cutaneous mite infestations by enhancing the absorption of sulfur and sulfur derivatives into mammalian skin. Preferably the embodiments of the present invention are useful in treatment and prevention of Demodex mites, such as Demodex folliculorum and Demodex brevis on human skin.

The compositions useful in the methods of the present invention are effective mitocide compositions comprising the following active ingredients: sulfur and sulfur derivatives.

Sulfur derivatives as used herein means any composition that contains organic or inorganic sulfides, inorganic sulfites, organic or inorganic mercaptans, or any other than is being applied to the skin or hair of a user, including but not limited to cationic sulfur compounds, such as selenium sulfide, potassium sulfide, poly-potassium sulfide, poly-calcium poly-sulfide, H₂S, sulfuric acid, bisulfides, sulfur dioxide, thiols, organic salts, sodium sulfacetamide, or combinations thereof (most preferably sodium sulfacetamide).

Sulfur (or elemental sulfur) is a chemically active element and there are several forms of elemental sulfur. Forms of elemental sulfur suitable for use in the present invention are those forms of elemental sulfur that are known to be useful in dermatological compositions, including but not limited to, colloidal, coated, enrobed, entrapped, fumed, precipitated, washed and sublimed sulfur, milk of sulfur and flowers of sulfur. The preferred form of sulfur for use in the present invention is precipitated sulfur.

Inorganic sulfides suitable for use in connection with the present invention are those inorganic sulfides known to be useful in dermatological compositions and include, but are not limited to, selenium sulfide, sodium thiosulfate as well as those inorganic sulfides having the formula: RS, RSH, R₂S, RSSR, or RSSH, wherein R is an inorganic element that can bind ionically or covalently with sulfur.

Organic sulfides suitable for use in connection with the present invention are those organic sulfides known to be useful in dermatological compositions and include, but are not limited to, those organic sulfides having the formula: RS, R2S, RSH, R′SSR′, or R′SSH, wherein R′ is an organic compound and its salts that can bind ionically or covalently with sulfur. Exemplary organic sulfides include, but are not limited to sodium thioglycolate (sodium mercaptoacetic acid), and gluathione.

Inorganic sulfites suitable for use in the present invention are those inorganic sulfites known to be useful in dermatological compositions, including but not limited to, sulfites and metabisulfites.

The carrier for active ingredients must be “dermatologically acceptable” in the sense of being compatible with the delivery of the active ingredients and not injurious to the subject. Carriers include those suitable for topical administration and may be prepared by methods known in the art.

In one embodiment, a composition of sulfur, sulfur derivatives, and a dermatologically acceptable carrier, with a pH of from about 6.5 to about 8.1 is topically applied to the effected skin. The sulfur derivatives may generally be present at about 1% to about 20%, preferably present at about 2% to about 15% and more preferably from about 5% to about 10% by weight (all percentages given are by weight). Generally sulfur is present from about 0.1% to about 20%, preferably from about 0.25% to about 10%, more preferably from about 1% to about 5%, and most preferably about 5% of the composition.

The composition may take the form of cleansers, foundations, creams, lotions, bars, powders, suspensions, gels, oils, milks, high sorption bases, solutions in cream form, mousses, and foams. The cleansers include but are not limited to masks, make-up removers, hydrating products, exfoliating agents, foaming cleansers, non-foaming cleansers, lotions, foaming detergent aqueous gels and oils, rinsable cleaning anhydrous gels, milks for removing make-up, and foaming creams (preferably soap-based). The composition is preferably a cleanser, most preferably a cleanser with an aqueous base, which has been found to kill about 38% to 45.2% of the mites initially present on the skin. The reduction includes both removing live and dead mites from the skin and killing mites that remain on the skin.

When the invention is embodied in a cleanser, e.g. a foaming cleanser or a mask, the invention provides easy removal of the cleanser and the suspended skin residue, including but not limited to cutaneous mites. Although it is difficult to deposit an active drug by means of a cleanser due to the short contact time, cleansing action and rinse-off inherent in the use of cleansers, the present invention surprisingly deposits sufficient active drug to reduce cutaneous mites and provides enhanced absorption of the sulfur and sulfur derivatives into the skin.

Cleanser compositions of the present invention can deposit the sulfur and sulfur derivatives and provide absorption into the skin even when the applied cleanser is rinsed more than once after application. Further, the cleanser composition may be applied repeatedly, such as by using, rinsing and rinsing again. This double application of the cleanser deposits more of the active drug on the skin than the single application and may be more effective in reducing mites.

The sulfur in the cleanser composition of this invention is preferably present at about 5%. About 25% of the sulfur and sulfur derivatives in the composition delivered is absorbed in the stratum corneum, epidermis, dermis, or any combination thereof by this method.

It is believed that the present invention may operate at least in part by the conversion on or within the skin of the sulfur and/or sulfur derivatives into other active forms of sulfur. This may take place in the stratum corneum or epidermis.

Another embodiment of the invention is a composition comprising water, xanthan gum, magnesium aluminum silicate, kaolin, silicone dioxide, sodium sulfacetamide, sodium thiosulfate, glyceryl stearate, PEG-100 Stearate, quillaia saponaria extract, benzyl alcohol, and sulfur.

Examples 1, 2 and 3, which follow, are embodiments of the present invention.

### EXAMPLE 1

A topical solution in cream form according to the present invention was prepared according to the following formula:

TABLE 1

| CTFA Name | Percent w/w |
|---|---|
| Purified Water USP | 50.51 |
| Sulfacetamide Sodium USP | 11.24 |
| Propylene Glycol | 8.00 |
| Isopropyl Myristate NF | 6.00 |
| Sulfur Precipitated USP | 5.00 |
| Light Mineral Oil | 5.00 |
| Polysorbate 60 | 3.40 |
| Sorbitan Monostearate NF | 2.10 |
| Cetyl Alcohol NF | 1.80 |
| Hydrogenated Coco- | 1.30 |
| glycerides | |
| Stearyl Alcohol NF | 1.20 |
| Ordenone | 1.00 |
| Benzyl Alcohol NF | 1.00 |
| Glyceryl Stearate & PEG-100 | 0.85 |
| Stearate | |
| Zinc Ricinoleate | 0.50 |
| Dimethicone | 0.50 |
| Xanthan Gum NF | 0.30 |
| Disodium EDTA USP | 0.10 |
| Sodium Thiosulfate USP | 0.10 |
| Fragrance | 0.10 |

### EXAMPLE 2

A cleanser according to the present invention was prepared according to the following formula:

TABLE 2

| CTFA Name | Percent w/w |
|---|---|
| Sodium Methyl Oleyltaurate | 9.00 |
| Disodium Oleamido MEA | 5.00 |
| Sulfosuccinate | |
| PEG-55 Propylene Glycol | 0.80 |
| Oleate | |
| Purified Water USP | 51.88 |
| Sodium Cocoyl Isethionate | 8.50 |
| Methyl Paraben NF | 0.15 |
| Propyl Paraben NF | 0.05 |
| Disodium EDTA NF | 0.10 |
| Cetyl Alcohol NF | 3.50 |
| Stearyl Alcohol | 1.50 |
| Glyceryl Stearate & PEG-100 | 2.50 |
| Stearate | |
| BHT | 0.10 |
| Sodium Thiosulfate USP | 0.10 |
| Sulfacetamide Sodium USP | 11.24 |
| Magnesium Aluminum | 0.40 |
| Silicate NF | |
| Xanthan Gum NF | 0.08 |
| Sulfur Precipitated USP | 5.00 |
| Fragrance 27160 | 0.10 |

### EXAMPLE 3

The present invention was embodied in a high sorption base of the following formula:

TABLE 3

| CTFA Name | Percent w/w |
|---|---|
| Phase A | |
| Purified Water | 41.76 |
| Xanthan Gum NF | 0.30 |
| Phase B | |
| Kaolin USP | 18.00 |
| Silicon Dioxide NF | 5.00 |
| Sulfacetamide Sodium USP | 11.29 |
| Sodium Thiosulfate | 0.10 |
| Phase C | |
| Glyceryl Stearate & PEG-100 | 10.00 |
| Stearate | |
| Quillaia Saponaria Extract | 1.00 |
| Benzyl Alcohol NF | 1.00 |
| Phase D | |
| Precipitated Sulfur USP | 5.00 |
| Phase E | |
| Witch Hazel (14% Alcohol) | 5.00 |
| Fragrance 27160 | 0.05 |

Each phase was compounded separately and then the phases were compounded together to give a finished product.

A high sorption base is a composition that contains ingredients (such as swelling clays and non-swelling clays) that act to absorb certain irritants such as sweat, and epidermal metabolites, from the skin.

Examples 4 and 5, which follow, describe the use embodiments of the present invention to demonstrate the delivery of sulfur to the skin layers.

### EXAMPLE 4

A topical solution in cream form according to Example 1 containing 5% radiolabeled sulfur (S³⁵) in addition to sodium sulfacetamide was applied at real-life use levels to the surface of wetted excised human skin mounted in a skin penetration cell. After 12 hours, the skin was rinsed and wiped off, and a second dose was then applied for an additional 12 hours. Then, the radiolabeled sulfur was determined (a) on the surface of and within the stratum corneum; (b) within the epidermis and within the dermis; and (c) within the reservoir (the reservoir was designed to emulate the blood circulation below the skin) underneath the skin which represents the amount passing through the skin.

The present invention in another embodiment may be used to deliver sulfur below the dermis and epidermis and systemically as evidenced in the data regarding the reservoir.

In this example, radiolabelled sodium sulfacetamide was not available but it is known sodium sulfacetamide has microbiological cutaneous activity.

The following table shows the results of clinical tests using the composition of this example:

TABLE 4
Micrograms of Radiolabeled Sulfur Deposited

| On the Stratum | Within the | | | |
|---|---|---|---|---|
| Corneum | Stratum | In the | In | |
| Surface | Corneum | Epidermis | the Dermis | In the Reservoir |
| 1344 | 295 | 117 | 27 | 26 |

Over 25% of the dose of sulfur deposited on the skin has been absorbed below the surface of the stratum corneum. These are important areas because lesions and inflammation occur in the stratum corneum, epidermis and dermis, and the composition is useful for treatment and prevention of lesions and inflammation. Dermal inflammation from mite infestations tends to reach into the dermis, therefore it is beneficial that the treatment in this embodiment penetrate into the dermis.

### EXAMPLE 5

A cleanser, according to Example 2 containing radiolabeled sulfur (S³⁵), and sodium sulfacetamide, was applied to the surface of wetted excised human skin mounted in a skin penetration cell. The applied cleanser was “massaged on the surface” for twenty seconds. Twelve cells were prepared. Then, in the first set of six cells, the cleanser was rinsed-off with water and wiping once. In the second set of six cells, the water rinse-off and wiping were performed two times in succession to create a further challenge to the deposition of sulfur. These two rinse-off regimens took place, in each cell, at zero time and then again 12 hours later. At 24 hours, the skin was removed from each cell and the amount of radio-labeled sulfur was determined: (a) on the surface of and within the stratum corneum; (b) within the epidermis and within the dermis; and (c) within the reservoir underneath the skin which represents the amount passing through the skin.

The following table shows the results of clinical tests using the composition of this example:

TABLE 5
Micrograms of Sulfur-35

| | On the | Within the | | | |
|---|---|---|---|---|---|
| Plexion | Stratum | Stratum | In the | In the | In the |
| Cleanser | Corneum | Corneum | Epidermis | Dermis | Reservoir |
| Rinsed Off | 40.3 | 9.2 | 9.2 | 3.3 | 1.7 |
| Once | | | | | |
| Rinsed Off | 5.3 | 6.7 | 8.0 | 1.8 | 2.1 |
| Twice | | | | | |

This data shows that this invention effectively delivers sulfur to and within the stratum corneum, epidermis and dermis following just 20 seconds of application, even after rigorous rinsing.

Further, while the second rinse and wipe-off reduced the amount of sulfur on the surface of the stratum corneum, it had less of an effect upon the amount of sulfur that penetrated into and was thus available for, efficacy within each skin compartment.

### EXAMPLE 6

Four high sorption formulations according to the present invention containing radiolabeled sulfur (S³⁵), in addition to sodium sulfacetamide, were applied at real-life use levels to the surface of wetted, excised human skin mounted in a skin penetration cell. Each treatment was left on the skin for 20 minutes before being rinsed and wiped off once. After 12 hours, a second dose of each formulation was then applied for an additional 20 minutes, then rinsed and wiped off once.

At 24 hours, the skin was removed from each cell and the amount of radio-labeled sulfur was determined: (a) on the surface of and within the stratum corneum; (b) within the epidermis and within the dermis; and (c) within the reservoir underneath the skin which represents the amount passing through the skin. The following table displays the data in micrograms of sulfur deposited and penetrating from the high sorption formulae.

TABLE 6

| | |
|---|---|
| CTFA Name | Percent w/w |

Formula A

| | |
|---|---|
| Purified Water | 41.76 |
| Xanthan Gum NF | 0.30 |
| Magnesium Aluminum Silicate | 1.50 |
| Kaolin USP | 18.00 |
| Silicon Dioxide NF | 5.00 |
| Sulfacetamide Sodium USP | 11.29 |
| Sodium Thiosulfate | 0.10 |
| Glyceryl Stearate & PEG-100 Stearate | 10.00 |
| Quillaia Saponaria Extract | 1.00 |
| Benzyl Alcohol NF | 1.00 |
| Precipitated Sulfur USP | 5.00 |
| Witch Hazel (14% alcohol) | 5.00 |
| Fragrance 27160 | 0.05 |

Formula B

| | |
|---|---|
| Distilled water | 41.76 |
| Xanthan Gum NF | 0.30 |
| Magnesium Aluminum Silicate | 1.50 |
| Glyceryl Stearate & PEG 100 Stearate | 10.00 |
| Quillaia Extract | 1.00 |
| Benzyl Alcohol | 1.00 |
| Kaolin USP | 18.00 |
| Silicon Dioxide | 2.00 |
| Sulfacetamide Sodium USP | 11.29 |
| Sodium Thiosulfate | 0.10 |
| Precipitated Sulfur USP | 5.00 |
| Silicon Dioxide | 3.00 |
| Witch Hazel (14% alcohol) | 5.00 |
| Fragrance 27160 | 0.05 |

Formula C

| | |
|---|---|
| Distilled Water | 46.76 |
| Xanthan Gum NF | 0.30 |
| Magnesium Aluminum Silicate | 1.50 |
| Glyceryl Stearate & PEG 100 Stearate | 10.00 |
| Quillaia Saponaria Extract (e.g. | 1.00 |
| Vegetol ® Bois de Panama) | |
| Benzyl Alcohol NF | 1.00 |
| Kaolin USP | 18.00 |
| Sulfacetamide Sodium USP | 11.29 |
| Sodium Thiosulfate | 0.10 |
| Precipitated Sulfur | 5.00 |
| Witch Hazel (14% alcohol) | 5.00 |
| Fragrance 27160 | 0.50 |

Formula D

| | |
|---|---|
| Distilled water | 46.76 |
| Xanthan gum NF | 0.30 |
| Magnesium Aluminum Silicate | 1.50 |
| Glyceryl Stearate & PEG 100 Stearate | 10.00 |
| Quillaia saponaria extract (e.g. | 1.00 |
| Vegetol ® Bois de Panama) | |
| Benzyl alcohol NF | 1.00 |
| Kaolin USP | 16.00 |
| Hectorite (e.g. Bentone ® 38) | 2.00 |
| Sulfacetamide Sodium USP | 11.29 |
| Sodium Thiosulfate | 0.10 |
| Precipitated Sulfur | 5.00 |
| Witch Hazel (14% alcohol) | 5.00 |
| Fragrance 27160 | 0.05 |

The following table displays the data in microorganisms of sulfur deposited and delivered by the four high sorption formulas.

| | On Stratum | Within the | | | |
|---|---|---|---|---|---|
| | Corneum | Stratum | In the | | In the |
| | Surface | Corneum | Epidermis | In the Dermis | Reservoir |
| A | 9.6 | 14.4 | 22.0 | 5.7 | 5.5 |
| B | 21.5 | 14.6 | 13.2 | 4.5 | 5.0 |
| C | 24.2 | 10.4 | 21.0 | 4.9 | 5.6 |
| D | 9.7 | 13.3 | 13.0 | 3.7 | 4.5 |

The sulfur released by all of the formulations was substantial and readily measurable. The sulfur levels released from these formulae are higher than in formulae for cleaners with the same sulfur content. The high sorption base has a longer residence time on the skin, than the cleanser before rinse-off.

Finally, except for the amounts of sulfur left on the stratum corneum's surface which were not absorbed or bioavailable, there is little difference between Formula A, and Formulae B, C and D. Thus, the high absorbency formula A did not impair the cutaneous bioavailability of sulfur.

Example 7 demonstrates the efficacy of embodiments of the present invention in treating mite infestations of the skin layers as compared to the efficacy of the prior art compositions.

### EXAMPLE 7

In this example, Demodex infestations were quantitatively determined by skin stripping the cheeks of human subjects three times with cyanoacrylate and counting the total mites in each strip. Four compositions were tested, the inventive composition from Example 1, the inventive composition from Example 2, Klaron® by Dermik Laboratories and Metrogel® by Galderma. Klaron's active drug is sodium sulfacetamide, and Metrogel's active drug is the pesticide metronidazole. Each product was tested on ten female and male subjects who had a minimum of at least one hundred mites in three skin strips in previous testing. Most subjects had between 200 and 500 mites per square meter.

Each product was applied twice daily to both cheek areas. Mites were counted from three skin strips on one side at Baseline and then from the other side after three weeks. The baseline and three-week cheeks, left and right, were randomized. The data represent the percent Increase (+) or Reduction (−) in mites.

The method for quantifying Demodex was as follows. A 3 cm adhesive ring was applied to the nasalar cheek, lateral to the tip of the nose. A drop of cyanoacrylate glue is applied and covered immediately with a plastic slide. After polymerization with in a few minutes, the slide is gently lifted off removing horny follicular casts within which the mites are encased. A second cyanoacrylate sample is taken from the same site. A drop of immersion oil is applied to the slide and the surface gently rubbed with a 29 gauge needle. This liberates the mites from the horny cocoons, allowing them to be counted under the stereomicroscope. The total count is the sum of the mites on the two specimens contained within the 3 cm adhesive ring.

For the Klaron® group, the average reduction in mites was 2.6%. For the Metrogel® group, the average reduction in mites was 11.9%. For the composition from Example 1, the average reduction in mites was 45.2%. For the composition from Example 2, the average reduction in mites was 38.0%.

TABLE 7

| | Average Reduction in Mites (Data Points |
|---|---|
| Product | listed in the Tables below) |
| Klaron ® | 2.6% |
| Metrogel ® | 11.9% |
| Example 1 | 45.2% |
| Example 2 | 38.0% |

The tables below reflect the actual data gathered in this example.

TABLE 8
Suppression of Demodex Infestation by Cleanser According to the
Invention Applied To Patients B.I.D. For Three Weeks

| | | Demodex Count | Demodex Count | |
|---|---|---|---|---|
| Subject | Age | Pre | Post | % Reduction |
| 1 | 38 | 191 | 140 | 27% |
| 2 | 45 | 602 | 433 | 28% |
| 3 | 45 | 448 | 327 | 29% |
| 4 | 50 | 521 | 290 | 44% |
| 5 | 42 | 462 | 314 | 32% |
| 6 | 43 | 524 | 319 | 39% |
| 7 | 37 | 202 | 116 | 42% |
| 8 | 46 | 528 | 315 | 40% |
| 9 | 52 | 456 | 231 | 49% |
| 10 | 56 | 555 | 299 | 46% |

TABLE 9
Suppression of Demodex Infestation by Topical Solution In Cream Form
According to the Invention
Applied To Patients B.I.D. For Three Weeks

| | | Demodex Count | Demodex Count | |
|---|---|---|---|---|
| Subject | Age | Pre | Post | % Reduction |
| 1 | 64 | 610 | 317 | 48% |
| 2 | 56 | 447 | 273 | 39% |
| 3 | 39 | 530 | 225 | 58% |
| 4 | 44 | 526 | 260 | 51% |
| 5 | 45 | 194 | 98 | 49% |
| 6 | 50 | 423 | 262 | 38% |
| 7 | 48 | 455 | 312 | 31% |
| 8 | 46 | 561 | 315 | 44% |
| 9 | 55 | 522 | 246 | 53% |
| 10 | 49 | 491 | 301 | 39% |

TABLE 10
Suppression of Demodex Infestation by 0.75% Metrogel
Applied To Patients B.I.D. For Three Weeks

| | | Demodex Count | Demodex Count | |
|---|---|---|---|---|
| Subject | Age | Pre | Post | % Reduction |
| 1 | 38 | 190 | 132 | 31% |
| 2 | 45 | 341 | 246 | 28% |
| 3 | 52 | 386 | 350 | 10% |
| 4 | 56 | 610 | 594 | 3% |
| 5 | 53 | 527 | 496 | 6% |
| 6 | 44 | 544 | 417 | 23% |
| 7 | 55 | 346 | 352 | −2% |
| 8 | 51 | 214 | 195 | 9% |
| 9 | 40 | 377 | 306 | 19% |
| 10 | 39 | 176 | 180 | −2% |

TABLE 11
Suppression of Demodex Infestation by Klaron Lotion
Applied To Patients B.I.D. For Three Weeks

| | | Demodex Count | Demodex Count | |
|---|---|---|---|---|
| Subject | Age | Pre | Post | % Reduction |
| 1 | 61 | 175 | 216 | −2% |
| 2 | 58 | 320 | 381 | 19% |
| 3 | 42 | 365 | 363 | 0.5% |
| 4 | 44 | 447 | 391 | 13% |
| 5 | 49 | 490 | 433 | 12% |
| 6 | 52 | 526 | 541 | −3% |
| 7 | 56 | 568 | 604 | −6% |
| 8 | 54 | 424 | 420 | 1% |
| 9 | 51 | 197 | 206 | 5% |
| 10 | 46 | 349 | 333 | 5% |

Example 7 shows that the present invention provides an effective treatment for mite infestations by dramatically reducing, or merely eliminating, mite populations in human skin.

It is observed that in addition to reduction or elimination of mite population the present invention also results in a significant reduction of inflammation and skin sensitization that is often assocaited with mite infestations.

It is understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed is:

1. A method for treatment of infestations of cutaneous mites comprising:
applying a composition to skin, wherein the composition comprises sulfur, one or more sulfur derivative and a dermatologically acceptable carrier.

2. The method of claim 1 further comprising washing the composition from the skin.

3. The method of claim 2 further comprising rewashing the composition from the skin.

4. The method of claim 1 wherein the composition comprises a cleanser.

5. The method of claim 1 wherein the composition has a pH of 6.5 to 8.1.

6. The method of claim 1, 2, or 3 wherein the composition has a pH of about 7.0 to about 8.1.

7. The method of claim 1, 2, or 3 wherein the composition has a pH of about 7.7 to about 8.1.

8. The method of claim 1, 2, or 3 wherein the composition has a pH of about 7.3 to about 7.7.

9. The method of claims 1, 2 or 3 wherein the composition comprises a cleanser with a pH of 6.5 to 8.1.

10. The method of claim 1 wherein the sulfur derivative comprises one or more cationic sulfur compounds.

11. The method of claim 1 wherein the sulfur derivative comprises one or more of the group consisting of selenium sulfide, potassium sulfide, poly-potassium sulfide, and poly-calcium poly-sulfide; H₂S; sulfuric acid; bisulfides; sulfur dioxide; thiols; organic salts; sodium sulfacetamide; sulfites; and mercaptans.

12. The method of claim 1 wherein the sulfur derivative comprises sodium sulfacetamide.

13. The method of claims 1, 2, or 3 wherein the composition comprises a cleanser with a pH of 6.5 to 8.1 and wherein the sulfur derivative comprises sodium sulfacetamide.

14. The method of claim 11 wherein the sulfur derivative is present in the range of about 10%.

15. The method of claims 1, 2, or 3 wherein the composition comprises a cleanser with a pH of 6.5 to 8.1 and wherein the sulfur derivative comprises sodium sulfacetamide present in the range of about 10% and wherein the sulfur is present in the range of about 5%.

16. The method of claim 1 wherein the mites are of the genus Demodex.

17. The method of claim 1 wherein the carrier comprises an aqueous base.

18. The method of claim 1 wherein the composition comprises a high sorption base.

19. The method of claim 18 wherein the high sorption base comprises one or more of the group consisting of non-swelling clay, gum, swelling clay and silicon.

20. The method of claim 19 wherein the non-swelling clay comprises kaolin.

21. The method of claim 19 wherein the non-swelling clay is present at about 18.00%.

22. The method of claim 19 wherein the gum comprises xanthan gum.

23. The method of claim 19 wherein the gum is present at about 0.30%.

24. The method of claim 19 wherein the swelling clay comprises magnesium aluminum silicate.

25. The method of claim 19 wherein the swelling clay is present at about 1.50%.

26. The method of claim 19 wherein the silicon comprises silicon dioxide.

27. The method of claim 19 wherein the silicon is present at about 5.00%.

28. The method of claim 19 wherein the composition further comprises water.

29. The method of claim 19 wherein the water is present at about 40-50%.

30. A method for prevention of infestations of cutaneous mites comprising
Applying a composition to skin, wherein the composition comprises sulfur, one or more sulfur derivative and a dermatologically acceptable carrier.

31. The method of claim 30 further comprising washing the composition from the skin.

32. The method of claim 31 further comprising rewashing the composition from the skin.

33. The method of claim 30 wherein the composition comprises a cleanser.

34. The method of claim 30, 31 or 32 wherein the composition has a pH of about 7.0 to about 8.1.

35. The method of claim 30, 31 or 32 wherein the composition has a pH of about 7.7 to about 8.1.

36. The method of claim 30, 31 or 32 wherein the composition has a pH of about 7.3 to about 7.7.

37. The method of claims 30, 31 or 32 wherein the composition comprises a cleanser with a pH of 6.5 to 8.1.

38. The method of claim 30 wherein the sulfur derivative comprises one or more cationic sulfur compounds.

39. The method of claim 30 wherein the sulfur derivative comprises one or more of the group consisting of selenium sulfide, potassium sulfide, poly-potassium sulfide, and poly-calcium poly-sulfide; H₂S; sulfuric acid; bisulfides; sulfur dioxide; thiols; organic salts; sodium sulfacetamide; sulfites; and mercaptans.

40. The method of claim 30 wherein the sulfur derivative comprises sodium sulfacetamide.

41. The method of claims 30, 31, or 32 wherein the composition comprises a cleanser with a pH of 6.5 to 8.1 and wherein the sulfur derivative comprises sodium sulfacetamide.

42. The method of claim 39 wherein the sulfur derivative is present in the range of about 10%.

43. The method of claims 30, 31, or 32 wherein the composition comprises a cleanser with a pH of 6.5 to 8.1 and wherein the sulfur derivative comprises sodium sulfacetamide present in the range of about 10% and wherein the sulfur is present in the range of about 5%.

44. The method of claim 30 wherein the mites are of the genus Demodex.

45. The method of claim 30 wherein the carrier comprises an aqueous base.

46. The method of claim 30 wherein the composition comprises a high sorption base.

47. The method of claim 46 wherein the high sorption base comprises one or more of the group consisting of non-swelling clay, gum, swelling clay and silicon.

48. The method of claim 47 wherein the non-swelling clay comprises kaolin.

49. The method of claim 47 wherein the non-swelling clay is present at about 18.00%.

50. The method of claim 47 wherein the gum comprises xanthan gum.

51. The method of claim 47 wherein the gum is present at about 0.30%.

52. The method of claim 47 wherein the swelling clay comprises magnesium aluminum silicate.

53. The method of claim 47 wherein the swelling clay is present at about 1.50%.

54. The method of claim 47 wherein the silicon comprises silicon dioxide.

55. The method of claim 47 wherein the silicon is present at about 5.00%.

56. The method of claim 47 wherein the composition further comprises water.

57. The method of claim 47 wherein the water is present at about 40-50%.

58. A method for treatment of infestations of cutaneous mites comprising:
Applying a composition to skin, wherein the composition comprises a cleanser comprising about 5% sulfur, about 10% sodium sulfacetamide and a dermatologically acceptable carrier, the composition has a pH of about 6.5 to 8.1;
Washing and rewashing the composition from the skin.

59. A method for reducing skin inflammation relating to infestations of cutaneous mites comprising
Applying a composition to skin, wherein the composition comprises sulfur, one or more sulfur derivative and a dermatologically acceptable carrier.

60. The method of claim 59 further comprising washing the composition from the skin.

61. The method of claim 60 further comprising rewashing the composition from the skin.

62. The method of claim 59 wherein the composition comprises a cleanser.

63. The method of claim 59, 60 or 61 wherein the composition has a pH of about 7.0 to about 8.1.

64. The method of claim 59, 60 or 61 wherein the composition has a pH of about 7.7 to about 8.1.

65. The method of claim 59, 60 or 61 wherein the composition has a pH of about 7.3 to about 7.7.

66. The method of claims 59, 60 or 61 wherein the composition comprises a cleanser with a pH of 6.5 to 8.1.

67. The method of claim 59 wherein the sulfur derivative comprises one or more cationic sulfur compounds.

68. The method of claim 59 wherein the sulfur derivative comprises one or more of the group consisting of selenium sulfide, potassium sulfide, poly-potassium sulfide, and poly-calcium poly-sulfide; H₂S; sulfuric acid; bisulfides; sulfur dioxide; thiols; organic salts; sodium sulfacetamide; sulfites; and mercaptans.

69. The method of claim 59 wherein the sulfur derivative comprises sodium sulfacetamide.

70. The method of claims 59, 60 or 61 wherein the composition comprises a cleanser with a pH of 6.5 to 8.1 and wherein the sulfur derivative comprises sodium sulfacetamide.

71. The method of claim 68 wherein the sulfur derivative is present in the range of about 10%.

72. The method of claims 59, 60 or 61 wherein the composition comprises a cleanser with a pH of 6.5 to 8.1 and wherein the sulfur derivative comprises sodium sulfacetamide present in the range of about 10% and wherein the sulfur is present in the range of about 5%.

73. The method of claim 59 wherein the mites are of the genus Demodex.

74. The method of claim 59 wherein the carrier comprises an aqueous base.

75. The method of claim 59 wherein the composition comprises a high sorption base.

76. The method of claim 75 wherein the high sorption base comprises one or more of the group consisting of non-swelling clay, gum, swelling clay and silicon.

77. The method of claim 76 wherein the non-swelling clay comprises kaolin.

78. The method of claim 76 wherein the non-swelling clay is present at about 18.00%.

79. The method of claim 76 wherein the gum comprises xanthan gum.

80. The method of claim 76 wherein the gum is present at about 0.30%.

81. The method of claim 76 wherein the swelling clay comprises magnesium aluminum silicate.

82. The method of claim 76 wherein the swelling clay is present at about 1.50%.

83. The method of claim 76 wherein the silicon comprises silicon dioxide.

84. The method of claim 76 wherein the silicon is present at about 5.00%.

85. The method of claim 76 wherein the composition further comprises water.

86. The method of claim 85 wherein the water is present at about 40-50%.

87. A method for reducing skin inflammation relating to infestations of cutaneous mites comprising
Applying a composition to skin, wherein the composition comprises sulfur, one or more sulfur derivative and a dermatologically acceptable carrier.

88. The method of claim 87 further comprising washing the composition from the skin.

89. The method of claim 88 further comprising rewashing the composition from the skin.

90. The method of claim 87 wherein the composition comprises a cleanser.

91. The method of claim 87, 88 or 89 wherein the composition has a pH of about 7.0 to about 8.1.

92. The method of claim 87, 88 or 89 wherein the composition has a pH of about 7.7 to about 8.1.

93. The method of claim 87, 88 or 89 wherein the composition has a pH of about 7.3 to about 7.7.

94. The method of claims 87, 88 or 89 wherein the composition comprises a cleanser with a pH of 6.5 to 8.1.

95. The method of claim 87 wherein the sulfur derivative comprises one or more cationic sulfur compounds.

96. The method of claim 87 wherein the sulfur derivative comprises one or more of the group consisting of selenium sulfide, potassium sulfide, poly-potassium sulfide, and poly-calcium poly-sulfide; H₂S; sulfuric acid; bisulfides; sulfur dioxide; thiols; organic salts; sodium sulfacetamide; sulfites; and mercaptans.

97. The method of claim 87 wherein the sulfur derivative comprises sodium sulfacetamide.

98. The method of claims 87, 88 or 89 wherein the composition comprises a cleanser with a pH of 6.5 to 8.1 and wherein the sulfur derivative comprises sodium sulfacetamide.

99. The method of claim 96 wherein the sulfur derivative is present in the range of about 10%.

100. The method of claims 87, 88 or 89 wherein the composition comprises a cleanser with a pH of 6.5 to 8.1 and wherein the sulfur derivative comprises sodium sulfacetamide present in the range of about 10% and wherein the sulfur is present in the range of about 5%.

101. The method of claim 87 wherein the mites are of the genus Demodex.

102. The method of claim 87 wherein the carrier comprises an aqueous base.

103. The method of claim 87 wherein the composition comprises a high sorption base.

104. The method of claim 103 wherein the high sorption base comprises one or more of the group consisting of non-swelling clay, gum, swelling clay and silicon.

105. The method of claim 103 wherein the non-swelling clay comprises kaolin.

106. The method of claim 103 wherein the non-swelling clay is present at about 18.00%.

107. The method of claim 103 wherein the gum comprises xanthan gum.

108. The method of claim 103 wherein the gum is present at about 0.30%.

109. The method of claim 103 wherein the swelling clay comprises magnesium aluminum silicate.

110. The method of claim 103 wherein the swelling clay is present at about 1.50%.

111. The method of claim 103 wherein the silicon comprises silicon dioxide.

112. The method of claim 103 wherein the silicon is present at about 5.00%.

113. The method of claim 103 wherein the composition further comprises water.

114. The method of claim 104 wherein the water is present at about 40-50%.

115. A method for prevention of infestations of cutaneous mites comprising:
Applying a composition to skin, wherein the composition comprises a cleanser comprising about 5% sulfur, about 10% sodium sulfacetamide and a dermatologically acceptable carrier, the composition has a pH of about 6.5 to 8.1;
Washing and rewashing the composition from the skin.
